(19) United States
(12) Patent Application Publication
(10) Pub. No.: US 2009/0270881 A1
(43) Pub. Date: Oct. 29, 2009

(54) FACIAL BLEMISH REMOVING DEVICE

(75) Inventor: Kevin M. CRUCS, Copley, OH (US)

Correspondence Address: HAHN LOESER & PARKS, LLP, One GOJO Plaza, Suite 300, AKRON, OH, 44311-1076, US

(73) Assignee: CRUCS HOLDINGS, LLC, Copley, OH (US)

(21) Appl. No.: 12/108,715

(22) Filed: Apr. 24, 2008

(51) Int. Cl. A61K 8/38; A61K 31/327; A61P 31/04; A61B 17/50; A45D 44/00

(52) U.S. Cl. 606/131; 514/714; 510/137; 604/290; 604/289

(57) ABSTRACT

A device and method for removing facial blemishes, including sebum that blocks facial pores. The device includes a substantially flat, elongated and rigid element having a first surface and a second surface, and a distal end and a proximal end. The device also includes a base member to which the rigid element is attached such that a collection area is formed between the second surface of the distal end of the rigid element and a portion of the base member.

### TECHNICAL FIELD

Embodiments of the present invention relate to a device and method for removing facial blemishes. More particularly, certain embodiments of the present invention relate to a device and method for removing sebum that blocks facial pores.

### BACKGROUND

The term “acne” is a general term to denote facial blemishes that are inflammatory disorders of the pilosebaceous unit. Acne is a group of disorders whose initial pathology is the comedo and includes acne vulgaris (common acne), neonatal acne, infantile acne, and pomade acne. Acne commonly afflicts adolescents and young adults, however, there is growing number of patients who develop acne in their late twenties or thirties. External agents that could exacerbate acne include medications e.g. iodides, anti-seizure, certain antibiotics and corticosteroids.

Acne is a chronic inflammatory disorder affecting the sebaceous glands. Acne lesions primarily involve the sebaceous glands located on the face, neck, chest and back. These lesions, including closed comedones (blackheads) and open comedones (whiteheads), are caused by hyperkeratinization of the infundibulum of the sebaceous duct. These keratinous plugs block the flow of sebum. These dilated ducts abound with the colonies of Propionibacterium acnes and other fat splitting organisms. The clinically evident open and closed comedones and the microscopic microcomedo are the signal lesions of acne. The acne process results from a cascade of events. First, at puberty a spike in androgen production heralds an increase in sebum production and begins the hyperkeratinization process causing microcomedones and sebum blockade. With this blockage, the number of resident follicular flora increases dramatically. These bacteria produce inflammatory products, which permeate through thin walls of dilated sebum-filled duct. Once in the perifollicular dermis, they trigger the body's own immune defenses (both acute and granulaomatous) to produce the characteristic inflammatory papules, pustules and nodules characteristic of inflammatory acne.

Increased sebum production; comedo formation, in which the follicular infundibulum hypercomifies, hyperkeratinizes, and hypodesquamates; colonization of the follicule by anaerobic Propionibacterium (mainly P. acnes); and the host's inflammatory response are generally believed to contribute to the development of acne. These four factors are interrelated. Sebum is comedogenic and causes inflammation by itself The Propionibacterium has high lipolytic activity and liberates free fatty acids from sebum lipids, whereby the free fatty acids have been shown to cause marked inflammation. The microorganisms also produce other extracellular enzymes such as proteases and hyaluronidases, and chemotactic factors, which may be important in the inflammatory process.

Acne treatments generally target the keratinous plugs in sebaceous ducts; large sebaceous glands producing excess sebum, increased numbers of resident follicular bacteria, and inflammatory response to chemical mediators passing through the follicular wall. Most acne treatments are directed at preventing inflammatory lesions, particularly the larger nodulo-cystic lesions which tend to be destructive and lead to permanent scarring. In general, visible comedones are the only minor cosmetic nuisances and do not lead to inflammatory lesions.

Continued blockage of these follicle openings causes a rupture of the bacteria into the dermis which, in turn, provokes an inflammatory response that leads to the formation of pimples. It is thus desirable to remove the unwanted cell layer (i.e., blackheads) from obstructed follicle openings in order to reduce inflammation and thereby eliminate the pimples.

Acne pads have been known and marketed for several years. Typically, a number of circular acne pads are soaked in a cleansing solution and then applied topically to the acne. The cleansing solution is typically a bactericide such as benzoyl peroxide which removes the unwanted cell layer obstructing the follicle openings by killing the bacteria. Although effective in treating acne, the cleansing solution applied via the acne pads may result in substantial irritation of the skin. Further, acne pads are often insufficient to remove blackheads embedded in the dermis.

Indeed, removing blackheads embedded in the dermis often requires the application of force to the affected area. Typically, two fingernail tips are pressed at complementary angles on opposite sides of the blackhead to pinch the blackhead outwards from the dermis. Although effective in exposing the underlying follicle opening, using one's fingernail tips to remove blackheads may inadvertently and undesirably cut surrounding areas of the dermis. In addition, when using one's fingernail tips to remove blackheads, bacteria and other germs residing under one's fingernails may come into contact with the freshly exposed follicle openings and/or any cuts on the dermis resulting from the pinching action of the fingernail tips.

Notwithstanding the state of the art as described herein, there is a need for further improvements in devices and methods treating facial blemishes, including various forms of acne, that clean the pores of the skin without the introduction of any further infection.

### BRIEF SUMMARY

Certain embodiments of the present invention pertain to a device for removing a facial blemish that includes a substantially flat, elongated and rigid element having a first surface and a second surface, and a distal end and a proximal end, and a base member, wherein the rigid element is attached to the base member such that a collection area is formed between the second surface of the distal end of the rigid element and a portion of the base member.

In one aspect of the embodiments of the present invention, the distal end of the rigid element is rounded.

In another aspect of the embodiments of the present invention, the base member has a shape selected from the group consisting of a substantially spherical shape and an ellipsoidal shape.

In yet another aspect of the embodiments of the present invention, the base member is comprised of a porous material.

In another aspect of the embodiments of the present invention, the base member is comprised of an absorbent material.

In yet another aspect of the embodiments of the present invention, the base member further includes a topical facial solution.

In still another aspect of the embodiments of the present invention, the facial solution includes an anti-bacterial solution.

In even another aspect of the embodiments of the present invention, the anti-bacterial solution includes a peroxide.

In still yet another aspect of the embodiments of the present invention, the peroxide is benzoyl peroxide.

In another of the embodiments of the present invention, the facial solution comprises a cleansing solution.

In still another aspect of the embodiments of the present invention, the cleansing solution loosens components of a blackhead blocking a sebaceous gland in the skin.

In yet another aspect of the embodiments of the present invention, the cleansing solution comprises water, iodine and Epsom salt.

In another of the embodiments of the present invention, the facial solution comprises an anti-bacterial solution and a cleansing solution.

In even another aspect of the embodiments of the present invention, the rigid element is fixedly attached to the base member.

In yet another aspect of the embodiments of the present invention, the rigid element is detachable to the base member.

In another aspect of the embodiments of the present invention, the device is contained within a hermetically sealed package prior to use.

In still another aspect of the embodiments of the present invention, the facial blemish is selected from the group consisting of whiteheads, blackheads, pimples and acne.

In yet even another aspect of the embodiments of the present invention, the facial blemish is a blackhead.

Another embodiment of the invention pertains to a method of removing a facial blemish, that includes the steps of providing a device for removing a facial blemish, the device including a substantially flat, elongated and rigid element having a first surface and a second surface, and a distal end and a proximal end, and a base member, wherein the rigid element is attached to the base member such that a collection area is formed between the second surface of the distal end of the rigid element and a portion of the base member, positioning the device between at least one user's finger and thumb, wherein the at least one finger rests on the top surface of the distal end of the rigid element and the thumb rests on a portion of the base member adjacent to the rigid element, contacting the first surface of the distal end of the rigid element and a bottom portion of the base member against a portion of a skin surface, advancing the device across the skin surface containing the facial blemish while maintaining contact against the skin, and extracting components of the facial blemish from at least one pore within the skin.

In another embodiment of the invention, the method further includes the step of collecting the components of the facial blemish in the collection area of the device as the device is advanced across the skin surface.

A further embodiment of the invention pertains to a method for removing sebum from blocked pores in a skin surface that includes the steps of providing a device for removing sebum from blocked pores in a skin surface, the device includes a substantially flat, elongated and rigid element having a first surface and a second surface, and a distal end and a proximal end, and a base member, wherein the rigid element is attached to the base member such that a collection area is formed between the second surface of the distal end of the rigid element and a portion of the base member, positioning the device between at least one user's finger and thumb, wherein the at least one finger rests on the top surface of the distal end of the rigid element and the thumb rests on a portion of the base member adjacent to the rigid element, contacting the first surface of the distal end of the rigid element and a bottom portion of the base member against a portion of a skin surface, advancing the device across the skin surface containing the sebum in the blocked pores of the skin surface while maintaining contact against the skin, extracting the sebum from the blocked pores of the skin surface, and collecting the sebum from blocked pores in a skin surface in the collection area of the device as the device is advanced across the skin surface.

These and other advantages and novel features, as well as details of an illustrated embodiment of the present invention, will be more fully understood from the following description and drawings.

### BRIEF DESCRIPTION OF THE DRAWINGS

FIG. 1 is a perspective view of an exemplary embodiment of a device for removing a facial blemish;

FIG. 2 is a bottom view of the device of FIG. 1;

FIG. 3 is a top view of another exemplary embodiment of a device for removing a facial blemish;

FIG. 4 is a bottom view of the device of FIG. 3;

FIG. 5 is a process diagram illustrating an exemplary embodiment of a method of use of the device of FIGS. 1-4 for removing a facial blemish; and

FIG. 6 is a side profile view of a typical user holding and using the device of FIGS. 1-4 for removing a facial blemish.

### DETAILED DESCRIPTION

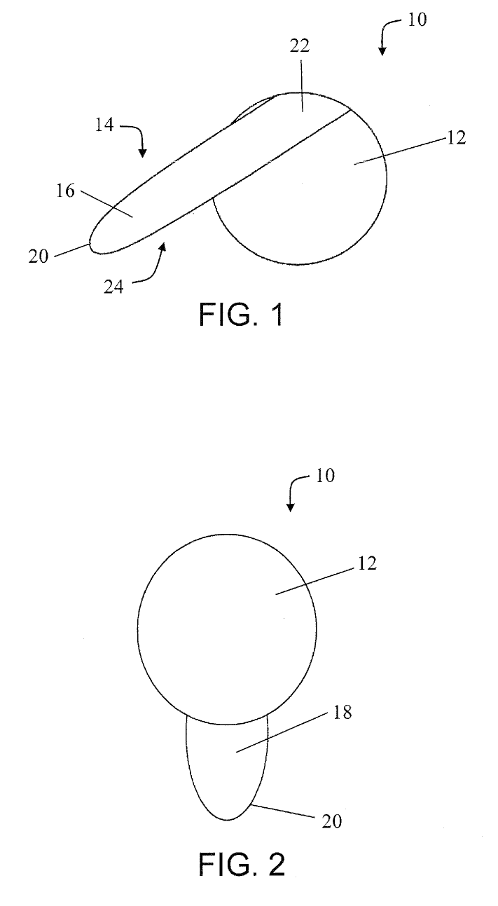

Referring now to the drawings wherein the showings are for purposes of illustrating certain embodiments of the present invention only and not for purposes of limiting the same, FIGS. 1-4 show a device 10 for removing a facial blemish. The device 10 includes a base member 12 and a rigid element 14 having a first surface 16, a second surface 18, a distal end 20 and a proximal end 22. The device 10 also includes a collection area 24 formed between a second or a bottom surface 18 of the distal end 20 of the rigid element 14 and a portion of the base member 12.

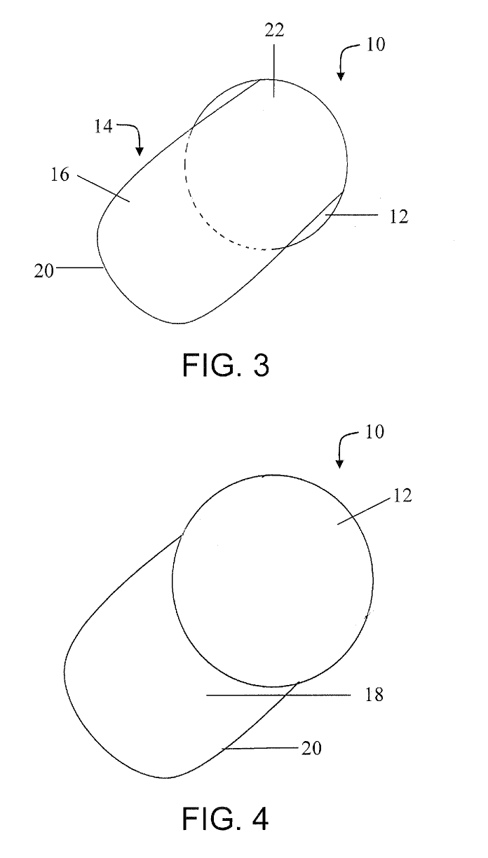

In one embodiment of the invention, the rigid element 14 is substantially flat and elongated. In another embodiment of the invention, the distal end 20 of the rigid element 14 is rounded as seen in FIGS. 1 and 2. In yet another embodiment of the invention, as seen in FIGS. 3 and 4, the distal end 20 of rigid element 14 is shaped substantially like a user's fingernail, that is, the distal end 20 has substantially rounded corner portions. The first surface 16 of the rigid element 14 may have serrations or grooves to aid in grasping the device 10. The rigid element 14 has a length adequate to be easily handled by a digit of a user on the first surface 16 and touches the surface of skin near the distal end 20. The rigid element 14 may be prepared from a material such as a metal or metal alloy, including those metals or metal alloys as used in dental or surgical equipment, or from a structural plastic material such as a thermoplastic or thermoset material. The material is safe for use in contact with the skin and strong enough so that it will not break when pressure is applied during normal use.

In another embodiment of the invention, the base member 12 may have a shape that is substantially spherical or ellipsoidal. In yet another embodiment of the invention, the base member 12 may be prepared from a non-porous, moldable material. An example of a non-porous, moldable material for base member 12 includes thermoplastic elastomers and polymers of ethylene and propylene, which includes polyethylene and polypropylene. In yet another embodiment of the invention, the base member 12 is made of a porous material, an adsorbent material or a porous and adsorbent material. In one embodiment, the porous and/or adsorbent materials include natural or synthetic sponges. The natural sponges are mostly marine, water dwelling, filter feeders found in oceans throughout the world, but can include cellulose wood fiber-based sponges. The synthetic sponges are prepared from foamed plastic polymers and include low-density polyether, which are known as non-absorbent sponges, polyvinyl alcohol-based sponges (PVA-based sponges), which are very dense, highly absorbent materials with no visible pores and polyester. The polyester sponges are also sub-divided into a variety of types including being reticulated (artificially broken-in) for ease of use and double-blown polyester, which have a high water retention ability, approaching or equaling PVA, but with visible pores and more flexibility of applications.

In another embodiment of the invention, the base member 12 includes a topical facial solution. The topical facial solution may include an anti-bacterial solution, a cleansing solution and mixtures thereof.

In one embodiment of the invention, the anti-bacterial solution includes a peroxide. In yet a further embodiment of the invention, the peroxide is benzoyl peroxide. In addition to its therapeutic effect as a keratolytic, which is a chemical that is capable of dissolving the sebum and keratin residing in the pores of the skin, benzoyl peroxide also prevents new lesions by killing the bacterium Propionibacterium acnes (P.acnes). In another embodiment of the invention, the anti-bacterial solution may include triclosan or chlorhexidine gluconate.

In yet another embodiment of the invention, the cleansing solution is a mixture of chemical agents designed to make the external layer of the skin, also known as the epidermis, as well as the pores of the epidermis, softer and more pliable. The cleansing solution also has the ability to loosen components of the facial blemish, including whiteheads and blackheads, prior to removal. The components loosened by the cleansing solution include dead skin cells, sebum, which is an oil secreted by the sebaceous gland within the skin, and dirt. In one embodiment, the cleansing solution includes water, iodine and Epsom salt.

The use of a peroxide in the anti-bacterial solution of the topical facial solution has the advantage of being a strong oxidizer which is essentially a mild bleach. However, this also has a tendency to routinely cause irritations such as dryness and redness of the skin to areas which the anti-bacterial solution has been applied. To overcome these irritations, the topical facial solution may include a moisturizer in the cleansing solution. In one embodiment, the cleansing solution includes naturally occurring skin lipids and sterols as well as artificial or natural oils, humectants, emollients, and lubricants.

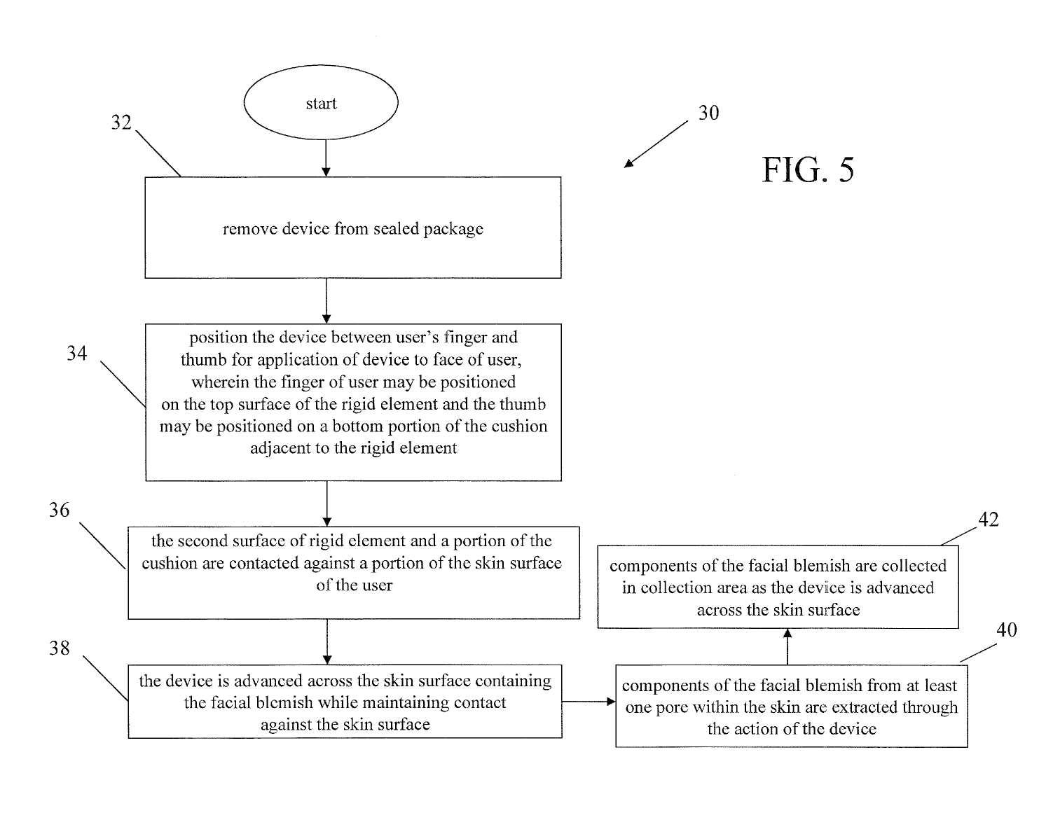

FIG. 5 illustrates a process diagram of an exemplary embodiment of a process for using device 10 for removing a facial blemish, such as a blackhead. Step 32 shows that the device 10 may be removed from a sealed package. Next, in step 34, the device 10 may be positioned between a user's finger, such as an index finger, and thumb for application to the face of the user. In particular, the finger of the user may be positioned on the first surface 16 of the distal end 20 of the rigid element 14 and the thumb may be positioned on a bottom portion of the base member 12 that is adjacent to the rigid element 14. Next, as described in step 36, the second surface 18 of the rigid element 14 and a portion of the base member 12 are contacted against a portion of the skin surface of the user. In step 38, the device 10 may be advanced across the skin surface containing the facial blemish while maintaining contact against the skin surface. Next, in step 40, components of the facial blemish from at least one pore within the skin may be extracted through the action of the device 10. Finally, as described in step 42, the components of the facial blemish may be collected in the collection area 24 as the device 10 is advanced across the skin surface of the user. After the components of the facial blemish have been collected, single-use device 10 may then be discarded.

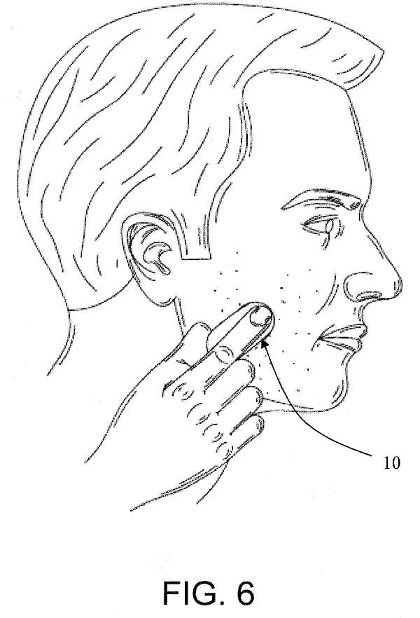

FIG. 6 illustrates a user holding and using device 10 on the skin surface of the face for removing a facial blemish, such as a blackhead.

In still yet another embodiment of the invention, device 10 is equipped with a securing means that is attached to first surface 16 of rigid element 14. For example, securing means may be a solid ring-shaped attachment, wherein the digit of a user is inserted into the ring-shaped attachment prior to use of removing a facial blemish with device 10. In another example, the securing means may be a plurality of hook and loop type fastening straps attached to first surface 16 of rigid element 14 or base member 12. In one embodiment, a first hook and loop type fastening strap is folded over the digit of a user as the digit rests on the first surface 16 of rigid element 14 and then a second hook and loop type fastening strap is folded onto the first hook and loop type fastening strap to secure the user's digit to device 10 prior to use of removing a facial blemish.

In yet another embodiment of the invention, device 10 is a multi-use device. In this embodiment, multi-use device 10 may be cleaned with soap, water and/or an alcohol, such as isopropyl alcohol, after the components of the facial blemish are collected. Device 10 may then be stored in an accessible location, such as a bathroom drawer for example, and be reused, as needed, at a future time.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiments disclosed, but that the invention will include all embodiments falling within the scope of the appended claims.

What is claimed is:

1. A device for removing a facial blemish, the device comprising:
a substantially flat, elongated and rigid element having a first surface and a second surface, and a distal end and a proximal end; and
a base member, wherein the rigid element is attached to the base member such that a collection area is formed between the second surface of the distal end of the rigid element and a portion of the base member.

2. The device of claim 1, wherein the distal end of the rigid element is rounded.

3. The device of claim 1, wherein the base member has a shape selected from the group consisting of a substantially spherical shape and an ellipsoidal shape.

4 The device of claim 1, wherein the base member is comprised of a porous material.

5 The device of claim 4 wherein the base member is comprised of an absorbent material.

6 The device of claim 4, wherein the base member further comprises a topical facial solution.

7 The device of claim 6, wherein the facial solution comprises an anti-bacterial solution.

8 The device of claim 7, wherein the anti-bacterial solution includes a peroxide.

9. The device of claim 8, wherein the peroxide is benzoyl peroxide.

10 The device of claim 6, wherein the facial solution comprises a cleansing solution.

11. The device of claim 10, wherein the cleansing solution comprises water, iodine and Epsom salt.

12 The device of claim 11, wherein the cleansing solution loosens components of a blackhead blocking a sebaceous gland in the skin.

13. The device of claim 6, wherein the facial solution comprises an anti-bacterial solution and a cleansing solution.

14. The device of claim 1, wherein the rigid element is fixedly attached to the base member.

15. The device of claim 1, wherein the rigid element is detachable to the base member.

16. The device of claim 1, wherein the device is contained within a hermetically sealed package prior to use.

17. The device of claim 1, wherein the facial blemish is selected from the group consisting of whiteheads, blackheads, pimples and acne.

18. The device of claim 1, wherein the facial blemish is a blackhead.

19. A method of removing a facial blemish, the method comprising the steps of:
providing a device for removing a facial blemish, the device comprising:
a substantially flat, elongated and rigid element having a first surface and a second surface, and a distal end and a proximal end; and
a base member, wherein the rigid element is attached to the base member such that a collection area is formed between the second surface of the distal end of the rigid element and a portion of the base member;
positioning the device between at least one user's finger and thumb, wherein the at least one finger rests on the top surface of the rigid element and the thumb rests on a portion of the base member adjacent to the rigid element;
contacting the first surface of the rigid element and a bottom portion of the base member against a portion of a skin surface;
advancing the device across the skin surface containing the facial blemish while maintaining contact against the skin surface; and
extracting components of the facial blemish from at least one pore within the skin surface.

20. The method of claim 19 further comprising the step of collecting the components of the facial blemish in the collection area of the device as the device is advanced across the skin surface.

21. The method of claim 19, wherein the distal end of the rigid element is rounded.

22. The method of claim 19, wherein the base member has a shape selected from the group consisting of a substantially spherical shape and an ellipsoidal shape.

23. The method of claim 19, wherein the base member is comprised of a porous material.

24. The method of claim 19, wherein the base member is comprised of an absorbent material.

25. The method of claim 19, wherein the base member further comprises a topical facial solution.

26. The method of claim 25, wherein the facial solution comprises an anti-bacterial solution.

27. The method of claim 26, wherein the anti-bacterial solution includes a peroxide.

28. The method of claim 27, wherein the peroxide is benzoyl peroxide.

29. The method of claim 25, wherein the facial solution comprises a cleansing solution.

30. The method of claim 29, wherein the cleansing solution loosens components of a blackhead blocking a sebaceous gland in the skin.

31. The method of claim 29, wherein the cleansing solution comprises water, iodine and Epsom salt.

32. The method of claim 25, wherein the facial solution comprises an anti-bacterial solution and a cleansing solution.

33. The method of claim 19, wherein the rigid element is fixedly attached to the base member.

34. The method of claim 19, wherein the rigid element is detachable to the base member.

35. The method of claim 19, wherein the device is contained within a hermetically sealed package prior to use.

36. The method of claim 19, wherein the facial blemish is selected from the group consisting of whiteheads, blackheads, pimples and acne.

37 The method of claim 36, wherein the facial blemish is a blackhead.

38. A method for removing sebum from blocked pores in a skin surface, the method comprising the steps of:
providing a device for removing sebum from blocked pores in a skin surface, the device comprising:
a substantially flat, elongated and rigid element having a first surface and a second surface, and a distal end and a proximal end; and
a base member, wherein the rigid element is attached to the base member such that a collection area is formed between the second surface of the distal end of the rigid element and a portion of the base member;
positioning the device between at least one user's finger and thumb, wherein the at least one finger rests on the top surface of the rigid element and the thumb rests on a portion of the base member adjacent to the rigid element;
contacting the first surface of the rigid element and a bottom portion of the base member against a portion of a skin surface;
advancing the device across the skin surface containing the sebum in the blocked pores of the skin surface while maintaining contact against the skin surface;
extracting the sebum from the blocked pores of the skin surface; and
collecting the sebum from blocked pores in a skin surface in the collection area of the device as the device is advanced across the skin surface.
